(19) United States
(12) Patent Application Publication
(10) Pub. No.: US 2019/0274876 A1
(43) Pub. Date: Sep. 12, 2019

(54) DEVICE AND METHOD FOR TEMPERATURE MODULATION OF THE NOSTRILS, NASAL CAVITY, AND ORAL ENTRANCE

(71) Applicant: CEDARS-SINAI MEDICAL CENTER, Los Angeles, CA (US)

(72) Inventor: Axel Rosengart, W. Hollywood, CA (US)

(73) Assignee: CEDARS-SINAI MEDICAL CENTER, Los Angeles, CA (US)

(21) Appl. No.: 16/345,195

(22) PCT Filed: Nov. 2, 2017
(86) PCT No.: PCT/US17/59741

§ 371 Date: Apr. 25, 2019

Related U.S. Application Data
(60) Provisional application No. 62/420,060, filed on Nov. 10, 2016.

(51) Int. Cl. A61F 7/12; F25D 5/02

(52) U.S. Cl. CPC A61F 7/12; A61F 2007/0008; F25D 5/02

(57) ABSTRACT

The disclosed devices and methods cool the nasal and/or oral cavity and perioral area, which in turn triggers a positive sensory reflex response to the brain leading to improvement of abnormally regulated cerebral vasomotor (brain arterial) tone which is thought to be the primary trigger factor for migraine headaches. Cooling is applied temporarily (10 to 30 minutes) to the inside of either or both nostrils. In some examples, the device is a single-use cooling “bullet” made from plastic (or other materials), has a conical, egg, nasal, oval, or other suitable shape and fits tightly to the inside of either nostril. The bullet may contain a nontoxic coolant (i.e., propylene glycol), water or other coolant which is kept at a cold state (e.g. 3-6 degrees Celsius below normal body temperature).

### FIELD

The present invention is directed to devices and methods for temperature modulation of the nostrils and nasal cavity, for example, useful in treating headaches.

### BACKGROUND

The following description includes information that may be useful in understanding the present invention. It is not an admission that any of the information provided herein is prior art or relevant to the presently claimed invention, or that any publication specifically or implicitly referenced is prior art.

Headaches of various etiologies, frequency and severity are very commonly reported and millions of people suffer from this illness. In addition to well-described treatments with headache medications and recommendations to apply physical methods as supplement treatment options (such as dark, quiet environment and cooling packs), a recent technology promotes cooled air to the nasal cavity. BeneChill is a company originally solely focusing on brain cooling technology especially brain cooling. However, in their clinical trials, patients who underwent brain cooling and simultaneously had headaches, experienced a great relief of head pain during nasopharyngeal cooling.

Specifically, BeneChill recently published a small trial using the following approach: “BeneChill's products consist of a portable control unit, a nasal catheter and a bottle of cooling fluid.” A maximum intranasal cooling period of 20 minutes was allowed per treatment; however, the average treatment time delivered was 15 minutes of cooling.” Interestingly, the success rate in this limited experience is very high compared to conventional headache treatment success rates (“Complete or partial pain relief was reported in 13 of the patients (87%)”).

### SUMMARY

In contrast to current treatment modalities (most commonly medications) the disclosed devices and methods cool the nostril and nasal cavity, which in turn triggers a positive sensory reflex response to the brain leading to improvement of abnormally regulated cerebral vasomotor (brain arterial) tone which is thought to be the primary trigger factor for migraine and other forms of headaches. Cooling is applied temporarily (i.e., 10 to 30 minutes) to the inside of either or both nostrils and nasal cavity.

In some examples, the device is a single-use cooling “bullet” made from plastic (or other materials), has a conical, egg, nasal, oval, or other suitable shape and fits tightly to the inside of either or both nostrils. The bullet may contain a nontoxic coolant (i.e., propylene glycol), water or other coolant which is kept at a cold or frozen state (i.e., 3-6 degrees Celsius or more below normal body temperature) and can be kept at target temperature in a household freezer (or refrigerator).

In some examples, the user will cool the bullet down to 3-6 degrees Celsius or more below the normal body temperature, and keep it at a target temperature in a household freezer (or refrigerator). After insertion into the nostril, in some examples the bullet maintains, for up to 30 minutes, a temperature profile within the range of 3-6 degrees Celsius below normal body temperature.

In some examples, only one bullet may be inserted into either nostril or one bullet into each nostril; the latter approach may use two individually bullet devices or combine two bullets via a holding bridge into one single device.

In some examples, cooling of the outer (skin) surface of the nose and surrounding upper lip or the addition of a cooling bite block for night-time use maybe employed to extend the surface cooling area; for example, the nasal cooling device can be connected to and be separate from a nasal cooling clip which fits onto the nose.

The bullet can be manufactured as a single-use or multi-use device. For the bullet to maintain its target temperature, it can be kept either cooled prior to usage or, alternatively, be activated by the release of pressurized, nontoxic gas or a (pressure-) activated chemical reaction originating from within the device. Optionally, the bullet can be coated with a medication (i.e., anesthetic; pain reliever, etc.) and/or a scent and/or, in case of an oral cooling device, with a flavor.

In contrast to available technologies, the proposed method/device is more mobile, more consumer-friendly, cheaper, simpler, allows single- or multi-use, easily storable and universally applicable by any layperson and without significant health risks from equipment failure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts, in accordance with various embodiments of the present invention, a perspective view of a cooling bullet;

FIG. 2 depicts, in accordance with various embodiments of the present invention, a flow chart illustrating a method of using the bullet in FIG. 1

FIG. 3 depicts, in accordance with various embodiments of the present invention, a perspective view of a cooling bullet with strap to retain in the patient's nostril.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

In contrast to current treatment modalities (most commonly medications) the disclosed devices and methods cool the nasal cavity, which in turn triggers a positive sensory reflex response to the brain leading to improvement of abnormally regulated cerebral vasomotor (brain arterial) tone which is thought to be the primary trigger factor for migraine headaches. In some examples, cooling may be applied for 10 to 30 minutes, 5 minutes, 15 minutes, 20 minutes, 40 minutes, one hour, or other suitable times to the inside of either or both nostrils and/or to the outer surface of the nose bridge and/or upper lip and/or oral cavity.

In some examples, the device is a single-use cooling “bullet” made from plastic (or other materials), and has a conical, egg, nasal, oval, or other suitable shape and fits tightly to the inside of either nostril. The bullet may contain a nontoxic coolant (i.e., propylene glycol), water or other coolant which is kept at a cold state—for example 3-6 degrees Celsius (or more) below normal body temperature—and can be kept at target temperature in a household freezer or refrigerator.

In some examples, the user will cool the bullet down to 3-6 degrees (or more) below the normal body temperature, and keep it at a target temperature in a household freezer (or refrigerator). After insertion into the nostril, in some examples the bullet maintains, for up to 30 minutes, a temperature profile within the range of 3-6 degrees Celsius below normal body temperature.

The bullet can be manufactured as a single-use or multi-use device. For the bullet to maintain its target temperature, it can be kept either cooled prior to usage or, alternatively, be activated by pressure and chemicals. Optionally, the bullet can be coated with a medication (i.e., anesthetic, pain medication, etc.) and/or a scent.

### Bullet Configuration

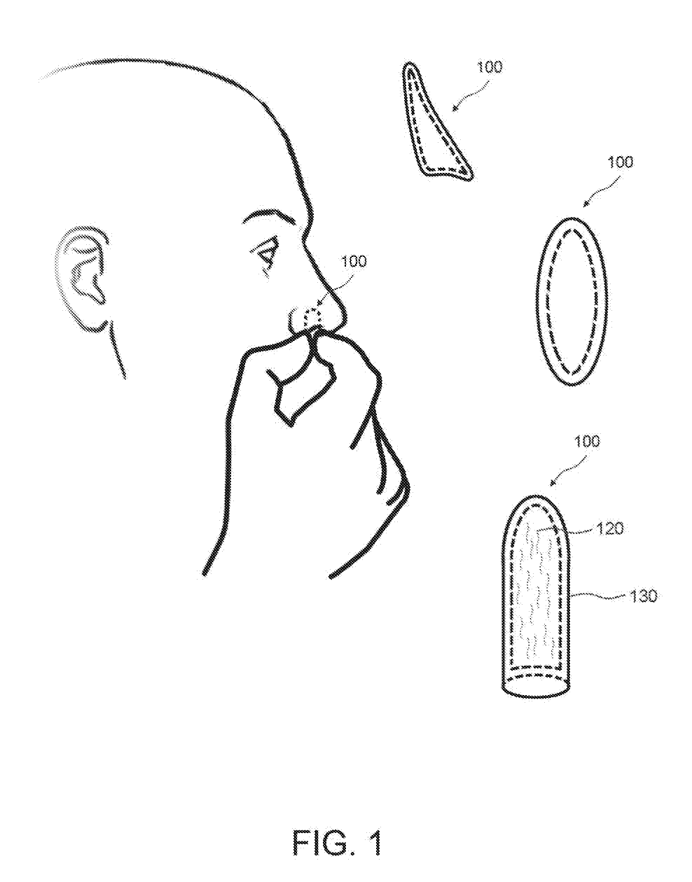

FIG. 1 illustrates a perspective view of several examples of a bullet 100 and its application inside the nostril. In some examples, the bullet 100 may include a case 130 and a coolant 120 inside the case 130.

The case 130 may be made from plastic, glass, ceramic, metal or other suitable materials. In some examples, the case 130 may be made of memory foam, so that the bullet 100 may be inserted and stay inside the nostril without the user holding it with their hand. In other examples, the case 130 may be made from wax, silicon, or other plastics. In other examples, the bullet 100 may be made of one solid piece or piece of memory foam. In some examples, the case 130 may have a central channel which allows airflow through the device and hence facilitates breathing.

The coolant 120 may be any suitable coolant 120 that has suitable thermal properties that allows the bullet to remain 3-6 degrees Celsius below body temperature for about 30 minutes or more when inserted into the nostril. For instance, the specific heat of the substance must be high enough, and may for instance be water (which has a very high specific heat).

In some cases, the coolant 120 may be a mixture of water and propylene glycol. In other examples, the mixture may be of water, hydroxyethyl cellulose, sodium polyacrylate, or vinyl-coated silica gel. The temperature gradient between the nostril and the bullet 100 ranges between minus 3 to 6 degrees Celsius or less, and therefore materials can be selected accordingly.

In some examples the coolant 120 and the case 130 will be one solid unitary unit and be made with a material that has a high specific heat. In some examples, the coolant 120 may use chemical endothermic reactions that instantly cool down the bullet 100, i.e., for single use devices. For instance, ammonium nitrate dissolving in water or urea dissolving in water results in an endothermic reaction. In this case, the coolant 120 may contain breakable bags, capsules or other delivery mechanisms to store the urea or ammonium nitrate. The user can then break the bags or capsules by manipulating the bullet 100 before application.

The bullet 100 may have any suitable shape that comfortably fits inside the nostril. For instance, the bullet 100 may be a classic bullet shape or may have a shape of a tear drop, ovoid, may have rounded ends, or may be tapered from a rounded point to a base to accommodate the shape of the nose. In some examples, the bullet 100 may come in different sizes, and may be sized so that it fits snuggly inside the nostril, but does not protrude below the nostril to avoid looking strange.

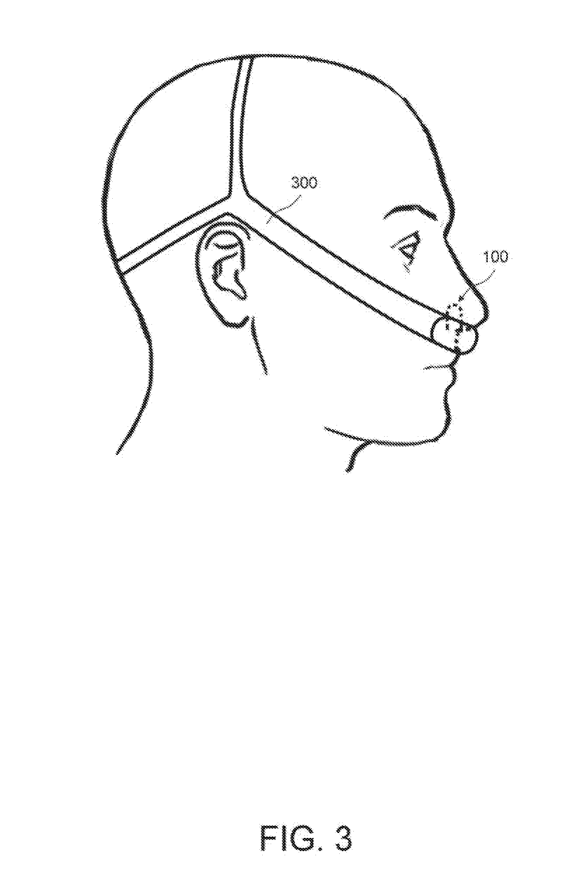

As illustrated in FIG. 3, the bullet 100 may be attached to a strap 300 that attaches around the patient's head. This way, the bullet 100 may be retained in the nostril without requiring the patient to hold it in their nostril. The strap 300 may connect to the bullet 100 with any attachment known in the art, or may have a pouch or other configuration that would cradle the end of the bullet 100 to retain it in the nostril. In some examples, the strap 300 may go through a loop or other opening at a base of the bullet 100.

### Applying Bullet in Nostril to Relieve Headache Pain

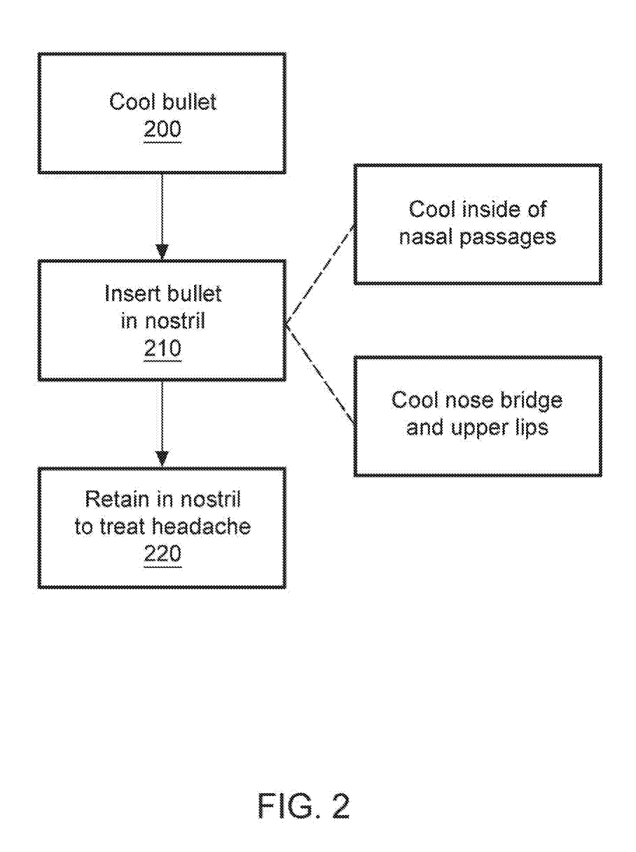

FIG. 2 illustrates a method of using the bullet 100 that includes first cooling the bullet 200 to an appropriate temperature, inserting the bullet in the nostril 210, and retaining the bullet in the nostril 220 for sufficient time. Then, the bullet 100 may be removed from the nostril and washed or sanitized for reuse, or discarded if it is single use.

First the bullet is cooled 200 to the appropriate temperature. This may include storing the bullet 100 in the refrigerator or freezer or leaving the bullet 100 at room temperature depending on the amount of temperature gradient desired. In some examples, the bullet may be first warmed to be 86 degrees Fahrenheit or 30 degrees Celsius (6 degrees below the normal body temperature of 36.5-37.5 degrees Celsius). A small thermo-strip placed at the outside of the device may indicate in a color-coded fashion the optimal time and temperature for device usage. In other examples, internal packaging may be broken to allow an endothermic reaction to cool the bullet 100.

Next, the bullet 100 will be inserted into the nostril 210. This may include by merely inserting the bullet 100 in the nostril and holding the bullet 100 in place with the user hand. In other examples, the bullet 100 may be form fitting to snuggly fit within the user's nostril. In other examples, the bullet 100 may include memory foam that a user may squeeze prior to putting it in the nostril, so that it expands and holds itself in place there. In other examples, the bullet 100 may have a central channel which allows airflow through the bullet.

It will then remain for a suitable amount of time 220 to reduce the user's headache pain or other symptoms from the headache. In some examples, this may include 10, 15, 20, 25, or 30 minutes, one hour or other suitable times. After using, the bullet 100 may be washed sterilized or thrown out. In some examples, the bullet 100 may include a casing 130 that can function as a sleeve of memory foam that may be discarded after each use, and a new sleeve engaged in the nose.

### Manufacturing and Materials

The bullet 100 may be manufactured from various materials and methods. For instance, if the casing is made of plastic, it may be injection molded, extruded and heat sealed, 3D printed or other various methods of manufacturing. In some examples, the casing 130 will be formed from plastic, and then the coolant 120 may be poured inside the casing 130 and then sealed inside. In some examples, 3D printing may be utilized to manufacture different layers with different specific heat capacities.

In some examples, the bullet 100 may be 3D printed to match the nasal cavity of a particular patient. For instance, a scan of the patient's nasal cavity may be converted into instructions for printing out the bullet in the shape of the patient's nasal cavity. In some examples, a form may be utilized by first inserting something pliable in a patient's nose, forming a mold, and then preparing a personalized bullet for one of the patient's nostril.

In some examples, there may be a bullet 100 personalized or sized for different nostrils, for instance for patients with deviated septums. In other examples, the bullet may come in shapes that are specialized to accommodate deviated septums.

### Selected Embodiments

Although the above description and the attached claims disclose a number of embodiments of the present invention, other alternative aspects of the invention are disclosed in the following further embodiments.

### Embodiment 1

A device for cooling of the nasal passages, perioral area and oral cavity, the device comprising:
a. a casing shaped and sized to fit within the nostril and contact the nose bridge and upper lip surface of a patient; and
b. a coolant retained inside the casing;

### Embodiment 2

The device of embodiment 1, wherein the casing is one of: bullet or ovoid shaped.

### Embodiment 3

The device of embodiment 1, wherein the casing is sized so as to be able to be contained entirely within a nostril.

### Embodiment 4

The device of embodiment 1, wherein the casing comprises at least one of: memory foam, plastic, water, or propylene glycol.

### Embodiment 5

The device of embodiment 1, wherein the coolant comprises of an energy-consuming chemical reaction.

### Embodiment 6

The device of embodiment 1, wherein a head strap is removably connected to the casing.

### Embodiment 7

The device of embodiment 6, wherein the casing comprising a loop through which the head strap is inserted.

### Embodiment 8

The device of embodiment 1, wherein the casing may have a central channel to allow airflow.

### Embodiment 9

A method of cooling the nasal passages to treat a headache, the method comprising:
a. providing the device of embodiment 1; and
b. inserting the device of embodiment 1 in the nostril of a patient for period of time sufficient to treat a headache.

### Embodiment 10

The method of embodiment 9, wherein the period of time is between 10 and 30 minutes.

### Embodiment 11

The method of embodiment 9, wherein the period of time is 15 minutes.

### Embodiment 12

The method of embodiment 9, wherein the device of embodiment 1 fits snuggly in the nostril and remains in the nostril using only force exerted on the bullet by the nostril walls.

### Embodiment 13

The method of embodiment 9, wherein the device of embodiment 1 is inserted so the casing fits entirely inside the nostril.

### Embodiment 14

A device for cooling of the nasal passages, the device comprising:
a. a casing shaped to contact the nose bridge and upper lip surface of a patient as a cooling bit block; and
b. a coolant retained inside the casing.

### Embodiment 15

The device of embodiment 14, wherein the casing is not configured to fit inside the nostril.

### Embodiment 16

A method of cooling the nasal passages to treat a headache, the method comprising:
a. providing a bullet;
b. cooling the bullet to below human body temperature; and
c. inserting the bullet inside the nostril of a patient for period of time sufficient to treat a headache.

### Embodiment 17

The method of embodiment 16, wherein the bullet is cooled 3-6 degrees Celsius below human body temperature.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A device for cooling of the nasal passages, perioral area and oral cavity, the device comprising:
a casing shaped and sized to fit within the nostril and contact the nose bridge and upper lip surface of a patient; and
a coolant retained inside the casing;

2. The device of claim 1, wherein the casing is one of: bullet or ovoid shaped.

3. The device of claim 1, wherein the casing is sized so as to be able to be contained entirely within a nostril.

4. The device of claim 1, wherein the casing comprises at least one of: memory foam, plastic, water, or propylene glycol.

5. The device of claim 1, wherein the coolant comprises of an energy-consuming chemical reaction.

6. The device of claim 1, wherein a head strap is removably connected to the casing.

7. The device of claim 6, wherein the casing comprising a loop through which the head strap is inserted.

8. The device of claim 1, wherein the casing may have a central channel to allow airflow.

9. A method of cooling the nasal passages to treat a headache, the method comprising:
providing the device of claim 1; and
inserting the device of claim 1 in the nostril of a patient for period of time sufficient to treat a headache.

10. The method of claim 9, wherein the period of time is between 10 and 30 minutes.

11. The method of claim 9, wherein the period of time is 15 minutes.

12. The method of claim 9, wherein the device of claim 1 fits snuggly in the nostril and remains in the nostril using only force exerted on the bullet by the nostril walls.

13. The method of claim 9, wherein the device of claim 1 is inserted so the casing fits entirely inside the nostril.

14. A device for cooling of the nasal passages, the device comprising:
a casing shaped to contact the nose bridge and upper lip surface of a patient as a cooling bit block; and
a coolant retained inside the casing.

15. The device of claim 14, wherein the casing is not configured to fit inside the nostril.

16. A method of cooling the nasal passages to treat a headache, the method comprising:
providing a bullet;
cooling the bullet to below human body temperature; and
inserting the bullet inside the nostril of a patient for period of time sufficient to treat a headache.

17. The method of claim 16, wherein the bullet is cooled 3-6 degrees Celsius below human body temperature.
